SEQUENCE LISTING (64 sequences)

SEQ ID NO: 1 - protein, 248 aa, synthetic construct
DILMTQSPLSLPVSLGDQASISCRSSQNIVHNNGITYLEWYLQRPGQSPKLLIYKVSDRFSGVPDRFSGSGSGTDFTLKISRVEAEDLGIYYCFQGSHIPPTFGGGTKLEIKRAAGGGGSGGGGSGGGGSQVQLQQSGSEMARPGASVKLPCKASGDTFTSYWMHWVKQRHGHGPEWIGNIYPGSGGTNYAEKFKNKVTLTVDRSSRTVYMHLSRLTSEDSAVYYCTRSGGPYFFDYWGQGTTLTVSS

SEQ ID NO: 2 - protein, 115 aa, synthetic construct
DILMTQSPLSLPVSLGDQASISCRSSQNIVHNNGITYLEWYLQRPGQSPKLLIYKVSDRFSGVPDRFSGSGSGTDFTLKISRVEAEDLGIYYCFQGSHIPPTFGGGTKLEIKRAA

SEQ ID NO: 3 - protein, 118 aa, synthetic construct
QVQLQQSGSEMARPGASVKLPCKASGDTFTSYWMHWVKQRHGHGPEWIGNIYPGSGGTNYAEKFKNKVTLTVDRSSRTVYMHLSRLTSEDSAVYYCTRSGGPYFFDYWGQGTTLTVSS

SEQ ID NO: 4 - protein, 16 aa, synthetic construct
RSSQNIVHNNGITYLE

SEQ ID NO: 5 - protein, 7 aa, synthetic construct
KVSDRFS

SEQ ID NO: 6 - protein, 9 aa, synthetic construct
FQGSHIPPT

SEQ ID NO: 7 - protein, 5 aa, synthetic construct
SYWMH

SEQ ID NO: 8 - protein, 17 aa, synthetic construct
NIYPGSGGTNYAEKFKN

SEQ ID NO: 9 - protein, 9 aa, synthetic construct
SGGPYFFDY

SEQ ID NO: 10 - protein, 203 aa, synthetic construct
APAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQ

SEQ ID NO: 11 - protein, 133 aa, synthetic construct
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 12 - protein, 19 aa, synthetic construct
MDWIWRILFLVGAATGAHS

SEQ ID NO: 13 - protein, 19 aa, synthetic construct
AEGRGSLLTCGDVEENPGP

SEQ ID NO: 14 - protein, 22 aa, synthetic construct
GSGATNFSLLKQAGDVEENPGP

SEQ ID NO: 15 - protein, 21 aa, synthetic construct
LCYLLDGILFIYGVILTALFL

SEQ ID NO: 16 - protein, 27 aa, synthetic construct
FWVLVVVGGVLACYSLLVTVAFIIFWV

SEQ ID NO: 17 - protein, 28 aa, synthetic construct
MFWVLVVVGGVLACYSLLVTVAFIIFWV

SEQ ID NO: 18 - protein, 22 aa, synthetic construct
MALIVLGGVAGLLLFIGLGIFF

SEQ ID NO: 19 - protein, 21 aa, synthetic construct
IYIWAPLAGTCGVLLLSLVIT

SEQ ID NO: 20 - protein, 23 aa, synthetic construct
IYIWAPLAGTCGVLLLSLVITLY

SEQ ID NO: 21 - protein, 24 aa, synthetic construct
IYIWAPLAGTCGVLLLSLVITLYC

SEQ ID NO: 22 - protein, 27 aa, synthetic construct
IISFFLALTSTALLFLLFFLTLRFSVV

SEQ ID NO: 23 - protein, 21 aa, synthetic construct
PFFFCCFIAVAMGIRFIIMVA

SEQ ID NO: 24 - protein, 10 aa, synthetic construct
GGGSSGGGSG

SEQ ID NO: 25 - protein, 12 aa, synthetic construct
ESKYGPPCPPCP

SEQ ID NO: 26 - protein, 12 aa, synthetic construct
ESKYGPPCPSCP

SEQ ID NO: 27 - protein, 22 aa, synthetic construct
ESKYGPPCPPCPGGGSSGGGSG

SEQ ID NO: 28 - protein, 39 aa, synthetic construct
IEVMYPPPYLDNEKSNGTIIHVKGKHLCPSPLFPGPSKP

SEQ ID NO: 29 - protein, 48 aa, synthetic construct
AKPTTTPAPRPPTPAPTIASQPLSLRPEACRPAAGGAVHTRGLDFACD

SEQ ID NO: 30 - protein, 45 aa, synthetic construct
TTTPAPRPPTPAPTIASQPLSLRPEACRPAAGGAVHTRGLDFACD

SEQ ID NO: 31 - protein, 129 aa, synthetic construct
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 229 aa, synthetic construct
ESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 229 aa, synthetic construct
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 41 aa, synthetic construct
RSKRSRLLHSDYMNMTPRRPGPTRKHYQPYAPPRDFAAYRS

SEQ ID NO: 37 - protein, 41 aa, synthetic construct
RSKRSRGGHSDYMNMTPRRPGPTRKHYQPYAPPRDFAAYRS

SEQ ID NO: 38 - protein, 42 aa, synthetic construct
KRGRKKLLYIFKQPFMRPVQTTQEEDGCSCRFPEEEEGGCEL

SEQ ID NO: 39 - protein, 42 aa, synthetic construct
ALYLLRRDQRLPPDAHKPPGGGSFRTPIQEEQADAHSTLAKI

SEQ ID NO: 40 - protein, 120 aa, synthetic construct
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 41-44 - skipped (4 entries)

SEQ ID NO: 45 - protein, 24 aa, synthetic construct
LEGGGEGRGSLLTCGDVEENPGPR

SEQ ID NO: 46 - skipped

SEQ ID NO: 47 - protein, 323 aa, synthetic construct
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 48-49 - skipped (2 entries)

SEQ ID NO: 50 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 51 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLYQGLSTATKDTYDALHMQALPPR

SEQ ID NO: 52 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLFQGLSTATKDTFDALHMQALPPR

SEQ ID NO: 53 - protein, 112 aa, synthetic construct
RVKFSRSADAPAYQQGQNQLYNELNLGRREEYDVLDKRRGRDPEMGGKPRRKNPQEGLYNELQKDKMAEAFSEIGMKGERRRGKGHDGLFQGLSTATKDTFDALHMQALPPR

SEQ ID NO: 54 - protein, 112 aa, synthetic construct
RVKFSRSADAPAYQQGQNQLYNELNLGRREEYDVLDKRRGRDPEMGGKPRRKNPQEGLFNELQKDKMAEAYSEIGMKGERRRGKGHDGLFQGLSTATKDTFDALHMQALPPR

SEQ ID NO: 55 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLYQGLSTATKDTFDALHMQALPPR

SEQ ID NO: 56 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTYDALHMQALPPR

SEQ ID NO: 57 - protein, 648 aa, synthetic construct
DILMTQSPLSLPVSLGDQASISCRSSQNIVHNNGITYLEWYLQRPGQSPKLLIYKVSDRFSGVPDRFSGSGSGTDFTLKISRVEAEDLGIYYCFQGSHIPPTFGGGTKLEIKRAAGGGGSGGGGSGGGGSQVQLQQSGSEMARPGASVKLPCKASGDTFTSYWMHWVKQRHGHGPEWIGNIYPGSGGTNYAEKFKNKVTLTVDRSSRTVYMHLSRLTSEDSAVYYCTRSGGPYFFDYWGQGTTLTVSSPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQ

SEQ ID NO: 58 - protein, 820 aa, synthetic construct
DILMTQSPLSLPVSLGDQASISCRSSQNIVHNNGITYLEWYLQRPGQSPKLLIYKVSDRFSGVPDRFSGSGSGTDFTLKISRVEAEDLGIYYCFQGSHIPPTFGGGTKLEIKRAAGGGGSGGGGSGGGGSQVQLQQSGSEMARPGASVKLPCKASGDTFTSYWMHWVKQRHGHGPEWIGNIYPGSGGTNYAEKFKNKVTLTVDRSSRTVYMHLSRLTSEDSAVYYCTRSGGPYFFDYWGQGTTLTVSSPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQAEGRGSLLTCGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 59 - protein, 17 aa, synthetic construct
PKSCDKTHTCPPCPDPK

SEQ ID NO: 60 - protein, 15 aa, synthetic construct
GGGGSGGGGSGGGGS

SEQ ID NO: 61 - protein, 10 aa, synthetic construct
GGGGSGGGGS

SEQ ID NO: 62 - protein, 22 aa, synthetic construct
MLLLVTSLLLCELPHPAFLLIP

SEQ ID NO: 63 - protein, 20 aa, synthetic construct
MYRMQLLSCIALSLALVTNS

SEQ ID NO: 64 - protein, 323 aa, synthetic construct
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

(19) United States
(12) Patent Application Publication
(10) Pub. No.: US 2025/0257138 A1
(43) Pub. Date: Aug. 14, 2025

(54) IMMUNE CELLS EXPRESSING A HYPOXIA-DEPENDENT CHIMERIC ANTIGEN RECEPTOR TARGETED TO EGFR

(71) Applicant: City of Hope, Duarte, CA (US)

(72) Inventors: Jianhua Yu, Duarte, CA (US); Michael A. Caligiuri, Pasadena, CA (US)

(21) Appl. No.: 18/963,261

(22) Filed: Nov. 27, 2024

Related U.S. Application Data
(60) Provisional application No. 63/603,759, filed on Nov. 29, 2023.

(51) Int. Cl. C07K 16/28; A61K 40/15; A61K 40/31; A61K 40/35; A61K 40/42; A61P 35/00; C07K 14/54; C12N 5/0783

(52) U.S. Cl. CPC C07K 16/2863; A61K 40/15; A61K 40/31; A61K 40/35; A61K 40/4204; A61K 40/4234; A61P 35/00; C07K 14/5443; C12N 5/0646; C07K 2317/73; C07K 2319/95; C12N 2510/00

(57) ABSTRACT

Described are immune cells expressing a chimeric antigen receptor targeted to EGFR and having an oxygen-dependent degradation domain. The chimeric antigen receptor is co-expressed in immune cells, e.g., NK cells with a soluble form of human IL-15. The inclusion of an oxygen-dependent degradation domain substantially restricts the activity of the immune cells expressing the chimeric antigen receptor to the more hypoxic environment of solid tumor tissue. The NK cells expressing the chimeric antigen receptor are useful for treating breast cancer that has low or no HER2 expression.

### CLAIM OF PRIORITY

This application claims the benefit of U.S. Provisional Application Ser. No. 63/603,759, filed on Nov. 29, 2023. The entire contents of the foregoing are incorporated herein by reference.

### SEQUENCE LISTING

This application contains a Sequence Listing which has been submitted electronically in XML format and is hereby incorporated by reference in its entirety. Said XML copy, created on Mar. 3, 2025, is named 40056-0089001_SL.xml and is 56,711 bytes in size.

### TECHNICAL FIELD

This disclosure relates to treating cancer using immune cells expressing a chimeric antigen receptor (CAR) targeted to epidermal growth factor receptor (EGFR) having an intracellular oxygen-dependent degradation domain (ODD). The CAR is co-expressed with human IL-15, e.g., a soluble form of human IL-15.

### BACKGROUND

Breast cancer is the most common cancer and the second leading cause of cancer death in women in North America, with an estimated 268,600 new cases of advanced breast cancer annually. Approximately 155,000 women are living with metastatic breast cancer in the United States with a median overall survival of 3.5 years. About 60-65% of breast cancers are estrogen receptor (ER) positive/human epidermal growth factor 2 (HER2) negative. More effective and less toxic therapies replacing or combined with conventional chemotherapy represent an area of unmet need in patients with HER2 negative metastatic breast cancer who have progressed through first- and second-line therapies.

HER2-low breast cancer is defined by HER2 immunohistochemistry 1+ or 2+ with negative fluorescence in situ hybridization. The clinical development of novel anti-HER2 agents has the potential to improve the treatment armamentarium for a subgroup of HER2-low breast cancer patients. Patients with HER2-low breast cancer are not currently considered candidates for HER2-targeted therapy. The HER2-low population accounts for up to 45-55% of all breast cancers. However, the anti-HER2 agents currently approved for the treatment of HER2-positive breast cancer have little or no activity in HER2-low tumors. Currently, there are limited treatment options for patients with HER2-low breast cancer, including estrogen receptor positive (ER+) breast cancer and triple-negative breast cancer (TNBC), which lacks expression of estrogen receptor (ER), progesterone receptor (PR), and HER2. This represents an area of unmet need in patients who progressed after initial targeted therapy.

### SUMMARY

Described herein are studies showing that, in some cases, EGFR is expressed or highly expressed on primary breast cancer tumor cells even when HER2 expression is at low levels. Also described are studies showing that EGFR is expressed on human MDA-MB-231, MDA-MB-468, and MCF-7 BC cell lines, as previously reported¹. To target the elevated expression of EGFR that we observed certain breast cancer tumor cells, we developed an EGFR-targeted CAR (EGFR-CAR) that includes an anti-EGFR single-chain variable fragment (scFv) targeting both wild-type EGFR and EGFRvIII. The CAR includes an anti-EGFR scFv, a spacer domain, a transmembrane domain, a CD28 co-stimulatory domain, and CD38. Given that IL-15 activates and improves the survival and activation of CD8 T cells and NK cells, we thus incorporated a soluble form of IL-15 (sIL-15) into the EGFR-CAR construct. We fused codon-optimized sIL-15 to EGFR-CAR using a Thosea asigna virus 2A-like ribosomal skip sequence (T2A). We linked a truncated, non-signaling form of CD19 (tCD19) to sIL-15 via porcine teschovirus-1 2A-like self-cleaving peptide (P2A). The tCD19 can serve as a marker to monitor CAR expression cells and can be used as a suicide gene switch with an anti-CD19 antibody. To reduce the activity of the CAR in non-cancer tissue (e.g., outside of the hypoxic tumor microenvironment), the EGFR-CAR is expressed as a fusion protein with the oxygen-dependent degradation domain (ODD) of HIF-1α. Solid tumor tissue is often hypoxic as a result of the high proliferation and high metabolic demand of tumor cells. Thus, tumor tissue can be below 5%, 4% or even below 2% O₂ (normal tissue is generally 5-15% O₂). The ODD of HIF1α directs degradation of HIF1α under hypoxic conditions. Under these conditions, prolines in the ODD are hydroxylated allowing the protein to be recognized by von Hippel-Lindau tumor suppressor, part of an E3 ubiquitin ligase complex that ubiquitinates HIF1α and targets it for proteasomal degradation.

When an ODD is included in a CAR, it directs degradation outside of tumor tissue where the O₂ level is higher. Overall the EGFR-CAR with an ODD and sIL-15 is referred to as EGFR-CAR_sIL15 (FIG. 2). Importantly, the EGFR-CAR_sIL15 is expressed in NK cells, which have allogeneic, “off-the-shelf” (not patient-specific) potential. NK cells can be manufactured from different sources such as peripheral blood cells, umbilical cord blood-derived NK cells, hematopoietic stem cells (e.g., CD34+ cells), induced pluripotent stem cells, etc.

The ODD can comprise or consist of the sequence: APAAGDTIISLDFGSNDTETDDQQLEEVPLYNDVMLPSPNEKLQNINLAMSPLPT AETPKPLRSSADPALNQEVALKLEPNPESLELSFTMPQIQDQTPSPSDGSTRQSSPE PNSPSEYCFYVDSDMVNEFKLELVEKLFAEDTEAKNPFSTQDTDLDLEMLAPYIP MDDDFQLRSFDQLSPLESSSASPESASPQSTVTVFQ (SEQ ID NO: 10). It can be followed by a ribosomal skip sequence, e.g.: AEGRGSLLTCGDVEENPGP (SEQ ID NO: 13).

The soluble IL-15 can comprise of consist of the sequence: GIHVFILGCFSAGLPKTEANWVNVISDLKKIEDLIQSMHIDATLYTESDVHPSCKV TAMKCFLLELQVISLESGDASIHDTVENLIILANNSLSSNGNVTESGCKECEELEE KNIKEFLQSFVHIVQMFINTS (SEQ ID NO:11), which can be preceded by a signal sequence that directs secretion from a mammalian cell, e.g.: MYRMQLLSCIALSLALVINS (SEQ ID NO: 63) and it can be followed by a ribosomal skip sequence, e.g., GSGATNFSLLKQAGDVEENPGP (SEQ ID NO: 14), which in turn can followed by a truncated CD19 receptor that acts both as a marker and a suicide switch. This truncated CD19 receptor can comprise or consist of the sequence:

(SEQ ID NO: 47)
MPPPRLLFFLLFLTPMEVRPEEPLVVKVEEGDNAVLQCLKGTSDG
PTQQLTWSRESPLKPFLKLSLGLPGLGIHMRPLAIWLFIFNVSQQ
MGGFYLCQPGPPSEKAWQPGWTVNVEGSGELFRWNVSDLGGLGCG
LKNRSSEGPSSPSGKLMSPKLYVWAKDRPEIWEGEPPCLPPRDSL
NQSLSQDLTMAPGSTLWLSCGVPPDSVSRGPLSWTHVHPKGPKSL
LSLELKDDRPARDMWVMETGLLLPRATAQDAGKYYCHRGNLTMSF
HLEITARPVLWHWLLRTGGWKVSAVTLAYLIFCLCSLVGILHLQR
ALVLRRKR.

The mature EGFR-CAR, with an ODD domain, can comprise or consist of the amino acid sequence:

(SEQ ID NO: 57)
DILMTQSPLSLPVSLGDQASISCRSSQNIVHNNGITYLEWYLQRP
GQSPKLLIYKVSDRFSGVPDRFSGSGSGTDFTLKISRVEAEDLGI
YYCFQGSHIPPTFGGGTKLEIKRAAGGGGSGGGGSGGGGSQVQLQ
QSGSEMARPGASVKLPCKASGDTFTSYWMHWVKQRHGHGPEWIGN
IYPGSGGTNYAEKFKNKVTLTVDRSSRTVYMHLSRLTSEDSAVYY
CTRSGGPYFFDYWGQGTTLTVSSPKSCDKTHTCPPCPDPKFWVLV
VVGGVLACYSLLVTVAFIIFWVRSKRSRLLHSDYMNMTPRRPGPT
RKHYQPYAPPRDFAAYRSRVKFSRSADAPAYQQGQNQLYNELNLG
RREEYDVLDKRRGRDPEMGGKPRRKNPQEGLYNELQKDKMAEAYS
EIGMKGERRRGKGHDGLYQGLSTATKDTYDALHMQALPPRAPAAG
DTIISLDFGSNDTETDDQQLEEVPLYNDVMLPSPNEKLQNINLAM
SPLPTAETPKPLRSSADPALNQEVALKLEPNPESLELSFTMPQIQ
DQTPSPSDGSTRQSSPEPNSPSEYCFYVDSDMVNEFKLELVEKLF
AEDTEAKNPFSTQDTDLDLEMLAPYIPMDDDFQLRSFDQLSPLES
SSASPESASPQSTVTVFQ.

When followed by a ribosomal skip sequence and sIL-15, the amino acid sequence of EGFR-CAR_sIL15 without the CAR signal sequence, but with the sIL-15 signal sequence, comprises or consists of:

(SEQ ID NO: 58)
DILMTQSPLSLPVSLGDQASISCRSSQNIVHNNGITYLEWYLQRP
GQSPKLLIYKVSDRFSGVPDRFSGSGSGTDFTLKISRVEAEDLGI
YYCFQGSHIPPTFGGGTKLEIKRAAGGGGSGGGGSGGGGSQVQLQ
QSGSEMARPGASVKLPCKASGDTFTSYWMHWVKQRHGHGPEWIGN
IYPGSGGTNYAEKFKNKVTLTVDRSSRTVYMHLSRLTSEDSAVYY
CTRSGGPYFFDYWGQGTTLTVSSPKSCDKTHTCPPCPDPKFWVLV
VVGGVLACYSLLVTVAFIIFWVRSKRSRLLHSDYMNMTPRRPGPT
RKHYQPYAPPRDFAAYRSRVKFSRSADAPAYQQGQNQLYNELNLG
RREEYDVLDKRRGRDPEMGGKPRRKNPQEGLYNELQKDKMAEAYS
EIGMKGERRRGKGHDGLYQGLSTATKDTYDALHMQALPPRAPAAG
DTIISLDFGSNDTETDDQQLEEVPLYNDVMLPSPNEKLQNINLAM
SPLPTAETPKPLRSSADPALNQEVALKLEPNPESLELSFTMPQIQ
DQTPSPSDGSTRQSSPEPNSPSEYCFYVDSDMVNEFKLELVEKLF
AEDTEAKNPFSTQDTDLDLEMLAPYIPMDDDFQLRSFDQLSPLES
SSASPESASPQSTVTVFQAEGRGSLLTCGDVEENPGPMYRMQLLS
CIALSLALVTNSGIHVFILGCFSAGLPKTEANWVNVISDLKKIED
LIQSMHIDATLYTESDVHPSCKVTAMKCFLLELQVISLESGDASI
HDTVENLIILANNSLSSNGNVTESGCKECEELEEKNIKEFLQSFV
HIVQMFINTS.

Thus, the mature proteins expressed on the surface of an immune cell (an NK cell, a T cell, an NKT cell, a macrophage, and gamma delta T cell) include:

(SEQ ID NO: 57)
DILMTQSPLSLPVSLGDQASISCRSSQNIVHNNGITYLEWYLQRP
GQSPKLLIYKVSDRFSGVPDRFSGSGSGTDFTLKISRVEAEDLGI
YYCFQGSHIPPTFGGGTKLEIKRAAGGGGSGGGGSGGGGSQVQLQ
QSGSEMARPGASVKLPCKASGDTFTSYWMHWVKQRHGHGPEWIGN
IYPGSGGTNYAEKFKNKVTLTVDRSSRTVYMHLSRLTSEDSAVYY
CTRSGGPYFFDYWGQGTTLTVSSPKSCDKTHTCPPCPDPKFWVLV
VVGGVLACYSLLVTVAFIIFWVRSKRSRLLHSDYMNMTPRRPGPT
RKHYQPYAPPRDFAAYRSRVKFSRSADAPAYQQGQNQLYNELNLG
RREEYDVLDKRRGRDPEMGGKPRRKNPQEGLYNELQKDKMAEAYS
EIGMKGERRRGKGHDGLYQGLSTATKDTYDALHMQALPPRAPAAG
DTIISLDFGSNDTETDDQQLEEVPLYNDVMLPSPNEKLQNINLAM
SPLPTAETPKPLRSSADPALNQEVALKLEPNPESLELSFTMPQIQ
DQTPSPSDGSTRQSSPEPNSPSEYCFYVDSDMVNEFKLELVEKLF
AEDTEAKNPFSTQDTDLDLEMLAPYIPMDDDFQLRSFDQLSPLES
SSASPESASPQSTVTVFQ
and
(SEQ ID NO: 11)
GIHVFILGCFSAGLPKTEANWVNVISDLKKIEDLIQSMHIDATLY
TESDVHPSCKVTAMKCFLLELQVISLESGDASIHDTVENLIILAN
NSLSSNGNVTESGCKECEELEEKNIKEFLQSFVHIVQMFINTS

In some cases, a mature, truncated CD19 receptor is also expressed by the cells. Described herein is a nucleic acid molecule encoding a fusion protein comprising a chimeric antigen receptor and an oxygen-dependent degradation domain (ODD), wherein the chimeric antigen receptor comprises: (i) an scFv that binds human EGFR; (ii) a spacer domain; (iii) a transmembrane domain; (iv) a costimulatory domain; and (v) a CD3ζ signaling domain.

In some embodiments: the scFv comprises: a light chain CDR1 comprising RSSQNIVHNNGITYLE (SEQ ID NO: 4), a light chain CDR2 comprising KVSDRFS (SEQ ID NO: 5), a light chain CDR3 comprising FQGSHIPPT (SEQ ID NO: 6), a heavy chain CDR1 comprising SYWMH (SEQ ID NO: 7), a heavy chain CDR2 comprising NIYPGSGGTNYAEKFKN (SEQ ID NO: 8), and a heavy chain CDR3 comprising SGGPYFFDY (SEQ ID NO: 9); the ODD is a human HIF1α ODD; the ODD comprises or consists of APAAGDTIISLDFGSNDTETDDQQLEEVPLYNDVMLPSPNEKLQNINLAMSPLPT AETPKPLRSSADPALNQEVALKLEPNPESLELSFTMPQIQDQTPSPSDGSTRQSSPE PNSPSEYCFYVDSDMVNEFKLELVEKLFAEDTEAKNPFSTQDTDLDLEMLAPYIP MDDDFQLRSFDQLSPLESSSASPESASPQSTVTVFQ (SEQ ID NO: 10); the scFv comprises a VL domain comprising: DILMTQSPLSLPVSLGDQASISCRSSQNIVHNNGITYLEWYLQRPGQSPKLLIYKV SDRFSGVPDRFSGSGSGTDFTLKISRVEAEDLGIYYCFQGSHIPPTFGGGTKLEIKR AA (SEQ ID NO: 2) with no more than 10, 5, 4, 3, 2 or 1 single amino acid substitutions (in some embodiments, the amino acid substitutions are not in the CDRs) followed by a VH domain comprising or consisting of QVQLQQSGSEMARPGASVKLPCKASGDTFTSYWMHWVKQRHGHGPEWIGNIY PGSGGTNYAEKFKNKVTLTVDRSSRTVYMHLSRLTSEDSAVYYCTRSGGPYFFD YWGQGTTLTVSS (SEQ ID NO: 3) with no more than 10, 5, 4, 3, 2 or 1 single amino acid substitutions (in some embodiments, the amino acid substitutions are not in the CDRs); the scFv comprises a VL domain comprising or consisting of an amino acid sequence at least 95%, 98% or 99% identical to: DILMTQSPLSLPVSLGDQASISCRSSQNIVHNNGITYLEWYLQRPGQSPKLLIYKV SDRFSGVPDRFSGSGSGTDFTLKISRVEAEDLGIYYCFQGSHIPPTFGGGTKLEIKR AA (SEQ ID NO: 2) with no more than 10, 5, 4, 3, 2 or 1 single amino acid substitutions (in some embodiments, the amino acid substitutions are not in the CDRs) followed by a VH domain comprising or consisting of an amino acid sequence at least 95%, 98% or 99% identical to QVQLQQSGSEMARPGASVKLPCKASGDTFTSYWMHWVKQRHGHGPEWIGNIY PGSGGTNYAEKFKNKVTLTVDRSSRTVYMHLSRLTSEDSAVYYCTRSGGPYFFD YWGQGTTLTVSS (SEQ ID NO: 3) with no more than 10, 5, 4, 3, 2 or 1 single amino acid substitutions (in some embodiments, the amino acid substitutions are not in the CDRs); the spacer region comprises an amino acid sequence selected from the group consisting of SEQ ID NOs: H and 24-34; the chimeric antigen receptor comprises: the spacer comprises or consists of a sequence selected from the group consisting of: SEQ ID NOs: H and 24-34; the transmembrane domain comprises or consists of a sequence selected from the group consisting of SEQ ID NOs: 15-23; the costimulatory domain comprises or consists of a sequence from the group consisting of SEQ ID NOs: 36-40, and the CD3 signaling domain comprises or consists of SEQ ID NO: 35; the nucleic acid molecule further encodes human IL-15 or a soluble variant of human IL-15; the nucleic acid molecule further encodes human IL-15 or a soluble variant of human IL-15 that is co-expressed with the fusion protein; the soluble variant of IL-15 comprises or consists of: GIHVFILGCFSAGLPKTEANWVNVISDLKKIEDLIQSMHIDATLYTESDVHPSCKV TAMKCFLLELQVISLESGDASIHDTVENLIILANNSLSSNGNVTESGCKECEELEE KNIKEFLQSFVHIVQMFINTS (SEQ ID NO:11) the nucleic acid molecule further includes a nucleotide sequence encoding signal sequence preceding the nucleotide sequence encoding SEQ ID NO: 11.

In some embodiments: the fusion protein comprises or consists of the amino acid sequence:

(SEQ ID NO: 57)
DILMTQSPLSLPVSLGDQASISCRSSQNIVHNNGITYLEWYLQRP
GQSPKLLIYKVSDRFSGVPDRFSGSGSGTDFTLKISRVEAEDLGI
YYCFQGSHIPPTFGGGTKLEIKRAAGGGGSGGGGSGGGGSQVQLQ
QSGSEMARPGASVKLPCKASGDTFTSYWMHWVKQRHGHGPEWIGN
IYPGSGGTNYAEKFKNKVTLTVDRSSRTVYMHLSRLTSEDSAVYY
CTRSGGPYFFDYWGQGTTLTVSSPKSCDKTHTCPPCPDPKFWVLV
VVGGVLACYSLLVTVAFIIFWVRSKRSRLLHSDYMNMTPRRPGPT
RKHYQPYAPPRDFAAYRSRVKFSRSADAPAYQQGQNQLYNELNLG
RREEYDVLDKRRGRDPEMGGKPRRKNPQEGLYNELQKDKMAEAYS
EIGMKGERRRGKGHDGLYQGLSTATKDTYDALHMQALPPRAPAAG
DTIISLDFGSNDTETDDQQLEEVPLYNDVMLPSPNEKLQNINLAM
SPLPTAETPKPLRSSADPALNQEVALKLEPNPESLELSFTMPQIQ
DQTPSPSDGSTRQSSPEPNSPSEYCFYVDSDMVNEFKLELVEKLF
AEDTEAKNPFSTQDTDLDLEMLAPYIPMDDDFQLRSFDQLSPLES
SSASPESASPQSTVTVFQ

with no more than 10, 5, 4, 3, 2, 1 or no single amino acid substitutions. In some embodiments, the amino acid substitutions are not in the targeting domain (e.g., the scFv, the VL domain and VH domain, and/or the CDRs).

In some embodiments: the nucleic acid molecule encodes an amino acid sequence comprising or consisting of the amino acid sequence:

(SEQ ID NO: 57)
DILMTQSPLSLPVSLGDQASISCRSSQNIVHNNGITYLEWYLQRP
GQSPKLLIYKVSDRFSGVPDRFSGSGSGTDFTLKISRVEAEDLGI
YYCFQGSHIPPTFGGGTKLEIKRAAGGGGSGGGGSGGGGSQVQLQ
QSGSEMARPGASVKLPCKASGDTFTSYWMHWVKQRHGHGPEWIGN
IYPGSGGTNYAEKFKNKVTLTVDRSSRTVYMHLSRLTSEDSAVYY
CTRSGGPYFFDYWGQGTTLTVSSPKSCDKTHTCPPCPDPKFWVLV
VVGGVLACYSLLVTVAFIIFWVRSKRSRLLHSDYMNMTPRRPGPT
RKHYQPYAPPRDFAAYRSRVKFSRSADAPAYQQGQNQLYNELNLG
RREEYDVLDKRRGRDPEMGGKPRRKNPQEGLYNELQKDKMAEAYS
EIGMKGERRRGKGHDGLYQGLSTATKDTYDALHMQALPPRAPAAG
DTIISLDFGSNDTETDDQQLEEVPLYNDVMLPSPNEKLQNINLAM
SPLPTAETPKPLRSSADPALNQEVALKLEPNPESLELSFTMPQIQ
DQTPSPSDGSTRQSSPEPNSPSEYCFYVDSDMVNEFKLELVEKLF
AEDTEAKNPFSTQDTDLDLEMLAPYIPMDDDFQLRSFDQLSPLES
SSASPESASPQSTVTVFQ

with no more than 10, 5, 4, 3, 2, 1 or no single amino acid substitutions; or the amino acid sequence:

(SEQ ID NO: 58)
DILMTQSPLSLPVSLGDQASISCRSSQNIVHNNGITYLEWYLQRP
GQSPKLLIYKVSDRFSGVPDRFSGSGSGTDFTLKISRVEAEDLGI
YYCFQGSHIPPTFGGGTKLEIKRAAGGGGSGGGGSGGGGSQVQLQ
QSGSEMARPGASVKLPCKASGDTFTSYWMHWVKQRHGHGPEWIGN
IYPGSGGTNYAEKFKNKVTLTVDRSSRTVYMHLSRLTSEDSAVYY
CTRSGGPYFFDYWGQGTTLTVSSPKSCDKTHTCPPCPDPKFWVLV
VVGGVLACYSLLVTVAFIIFWVRSKRSRLLHSDYMNMTPRRPGPT
RKHYQPYAPPRDFAAYRSRVKFSRSADAPAYQQGQNQLYNELNLG
RREEYDVLDKRRGRDPEMGGKPRRKNPQEGLYNELQKDKMAEAYS
EIGMKGERRRGKGHDGLYQGLSTATKDTYDALHMQALPPRAPAAG
DTIISLDFGSNDTETDDQQLEEVPLYNDVMLPSPNEKLQNINLAM
SPLPTAETPKPLRSSADPALNQEVALKLEPNPESLELSFTMPQIQ
DQTPSPSDGSTRQSSPEPNSPSEYCFYVDSDMVNEFKLELVEKLF
AEDTEAKNPFSTQDTDLDLEMLAPYIPMDDDFQLRSFDQLSPLES
SSASPESASPQSTVTVFQAEGRGSLLTCGDVEENPGPMYRMQLLS
CIALSLALVTNSGIHVFILGCFSAGLPKTEANWVNVISDLKKIED
LIQSMHIDATLYTESDVHPSCKVTAMKCFLLELQVISLESGDASI
HDTVENLIILANNSLSSNGNVTESGCKECEELEEKNIKEFLQSFV
HIVQMFINTS

with no more than 10, 5, 4, 3, 2, 1 or no single amino acid substitutions. In some embodiments, the amino acid substitutions are not in the targeting domain (e.g., the scFv, the VL domain and VH domain, and/or the CDRs).

Also disclosed is an immune cell (e.g., a T cell, an NK cell, an NKT cell, a macrophage, or a gamma delta T cell) harboring a nucleic acid described herein. In some embodiments: the immune cell expresses human IL-15 or soluble variant thereof; the soluble variant of IL-15 comprises or consists of: GIHVFILGCFSAGLPKTEANWVNVISDLKKIEDLIQSMHIDATLYTESDVHPSCKV TAMKCFLLELQVISLESGDASIHDTVENLIILANNSLSSNGNVTESGCKECEELEE KNIKEFLQSFVHIVQMFINTS (SEQ ID NO:11); and the immune cell is an NK cell.

Also described is a method for treating breast cancer comprising administering a composition comprising an immune cell described herein.

Also described is fusion protein comprising a chimeric antigen receptor and an oxygen-dependent degradation domain (ODD), wherein the chimeric antigen receptor comprises: (i) an scFv that binds human EGFR; (ii) a spacer domain; (iii) a transmembrane domain; (iv) a costimulatory domain; and (v) a CD3ζ signaling domain.

In various embodiment of the fusion protein: the scFv comprises: a light chain CDR1 comprising RSSQNIVHNNGITYLE (SEQ ID NO: 4), a light chain CDR2 comprising KVSDRFS (SEQ ID NO: 5), a light chain CDR3 comprising FQGSHIPPT (SEQ ID NO: 6), a heavy chain CDR1 comprising SYWMH (SEQ ID NO: 7), a heavy chain CDR2 comprising NIYPGSGGTNYAEKFKN (SEQ ID NO: 8), and a heavy chain CDR3 comprising SGGPYFFDY (SEQ ID NO: 9); the ODD is a human HIF1α ODD; the ODD comprises or consists of APAAGDTIISLDFGSNDTETDDQQLEEVPLYNDVMLPSPNEKLQNINLAMSPLPT AETPKPLRSSADPALNQEVALKLEPNPESLELSFTMPQIQDQTPSPSDGSTRQSSPE PNSPSEYCFYVDSDMVNEFKLELVEKLFAEDTEAKNPFSTQDTDLDLEMLAPYIP MDDDFQLRSFDQLSPLESSSASPESASPQSTVTVFQ (SEQ ID NO: 10); the scFv comprises a VL domain comprising: DILMTQSPLSLPVSLGDQASISCRSSQNIVHNNGITYLEWYLQRPGQSPKLLIYKV SDRFSGVPDRFSGSGSGTDFTLKISRVEAEDLGIYYCFQGSHIPPTFGGGTKLEIKR AA (SEQ ID NO: 2) with no more than 10, 5, 4, 3, 2 or 1 single amino acid substitutions followed by a VH domain comprising or consisting of QVQLQQSGSEMARPGASVKLPCKASGDTFTSYWMHWVKQRHGHGPEWIGNIY PGSGGTNYAEKFKNKVTLTVDRSSRTVYMHLSRLTSEDSAVYYCTRSGGPYFFD YWGQGTTLTVSS (SEQ ID NO: 3) with no more than 10, 5, 4, 3, 2 or 1 single amino acid substitutions; and the fusion protein comprises or consists of the amino acid sequence:

(SEQ ID NO: 57)
DILMTQSPLSLPVSLGDQASISCRSSQNIVHNNGITYLEWYLQRP
GQSPKLLIYKVSDRFSGVPDRFSGSGSGTDFTLKISRVEAEDLGI
YYCFQGSHIPPTFGGGTKLEIKRAAGGGGSGGGGSGGGGSQVQLQ
QSGSEMARPGASVKLPCKASGDTFTSYWMHWVKQRHGHGPEWIGN
IYPGSGGTNYAEKFKNKVTLTVDRSSRTVYMHLSRLTSEDSAVYY
CTRSGGPYFFDYWGQGTTLTVSSPKSCDKTHTCPPCPDPKFWVLV
VVGGVLACYSLLVTVAFIIFWVRSKRSRLLHSDYMNMTPRRPGPT
RKHYQPYAPPRDFAAYRSRVKFSRSADAPAYQQGQNQLYNELNLG
RREEYDVLDKRRGRDPEMGGKPRRKNPQEGLYNELQKDKMAEAYS
EIGMKGERRRGKGHDGLYQGLSTATKDTYDALHMQALPPRAPAAG
DTIISLDFGSNDTETDDQQLEEVPLYNDVMLPSPNEKLQNINLAM
SPLPTAETPKPLRSSADPALNQEVALKLEPNPESLELSFTMPQIQ
DQTPSPSDGSTRQSSPEPNSPSEYCFYVDSDMVNEFKLELVEKLF
AEDTEAKNPFSTQDTDLDLEMLAPYIPMDDDFQLRSFDQLSPLES
SSASPESASPQSTVTVFQ

with no more than 10, 5, 4, 3, 2, 1 or no single amino acid substitutions. In some embodiments, the amino acid substitutions are not in the targeting domain (e.g., the scFv, the VL domain and VH domain, and/or the CDRs).

### I. Chimeric Antigen Receptor

A chimeric antigen receptor (CAR) refers to an artificial immune cell receptor that is engineered to recognize and bind to a surface antigen. A NK cell that expresses a CAR polypeptide is referred to as a CAR NK cell. CARs have the ability to redirect immune cell specificity and reactivity toward a selected target in a non-MHC-restricted manner. The non-MHC-restricted antigen recognition gives CAR NK cells the ability to recognize an antigen independent of antigen processing, thereby bypassing a major mechanism of tumor escape. A CAR can also be expressed by other immune effector cells, including, but not limited to, T cells (“CAR T cells”) and direct T cell killing to cells expressing the target of the CAR.

There are various generations of CARs, each of which contains different components. First generation CARs join an antibody-derived scFv to the CD3 intracellular signaling domain of the T cell receptor through a spacer region (also called a hinge domain) and a transmembrane domain. Second generation CARs incorporate an additional co-stimulatory domain (e.g., CD28, 4-BB, or ICOS) to supply a co-stimulatory signal. Third generation CARs contain two co-stimulatory domains (e.g., a combination of CD27, CD28, 4-1BB, ICOS, or OX40) fused with the TCR CD3ζ chain. Any generation of CAR is within the scope of the present disclosure.

The CAR described herein are fusion proteins comprising an extracellular domain that recognizes a tumor cell antigen (e.g., a single chain fragment (scFv) of an antibody or other antibody fragment), a spacer, a transmembrane domain, at least one co-stimulatory domain and an intracellular domain comprising a signaling domain of the T cell receptor (TCR) complex (e.g., CD3ζ). A CAR is often fused to a signal peptide at the N-terminus for surface expression.

(a) Target Binding scFv

An EGFR CAR can include: (i) an scFv that binds EGFR; (ii) a spacer domain; (iii) a transmembrane domain; (iv) a costimulatory domain; and (v) a CD32 signaling domain. In various embodiments: the spacer region comprises 5-300 amino acids. The EGFR scFv can comprise: nucle The EGFR scFV can comprise: (a) a light chain variable domain that is at least 90%, 95% or 98% identical to: (SEQ ID NO: 2); and (b) a heavy chain variable domain that is at least 90%, 95% or 98% identical to: (SEQ ID NO:3). The light chain variable domain can preceded the heavy chain variable domain and they can be joined by a linker that includes 5-20 amino acids, preferably G and S. For example, the EGFR scFv can comprise or consist of: DILMTQSPLSLPVSLGDQASISCRSSQNIVHNNGITYLEWYLQRPGQSPKLLIYKV SDRFSGVPDRFSGSGSGTDFTLKISRVEAEDLGIYYCFQGSHIPPTFGGGTKLEIKR AAGGGGSGGGGSGGGGSQVQLQQSGSEMARPGASVKLPCKASGDTFTSYWMH WVKQRHGHGPEWIGNIYPGSGGTNYAEKFKNKVTLTVDRSSRTVYMHLSRLTS EDSAVYYCTRSGGPYFFDYWGQGTTLTVSS (SEQ ID NO:1). Thus, the scFv can include a VL domain DILMTQSPLSLPVSLGDQASISCRSSQNIVHNNGITYLEWYLQRPGQSPKLLIYKV SDRFSGVPDRFSGSGSGTDFTLKISRVEAEDLGIYYCFQGSHIPPTFGGGTKLEIKR AA (SEQ ID NO: 2) followed by a VH domain comprising or consisting of QVQLQQSGSEMARPGASVKLPCKASGDTFTSYWMHWVKQRHGHGPEWIGNIY PGSGGTNYAEKFKNKVTLTVDRSSRTVYMHLSRLTSEDSAVYYCTRSGGPYFFD YWGQGTTLTVSS (SEQ ID NO: 3). A linker having the sequence: GGGGSGGGGSGGGGS (SEQ ID NO: 60) or GGGGSGGGGS (SEQ ID NO: 61) can be located between the VH and VL domains.

The spacer region can comprise or consist of an amino acid sequence selected from the group consisting of SEQ ID NOs: H and 24-34; the transmembrane domain can comprise or consist of: PKSCDKTHTCPPCPDPK (SEQ ID NO: 59); a CD4 transmembrane domain, a CD8 transmembrane domain, a CD28 transmembrane a domain (either CD28 or CD28 (M)), and a CD32 transmembrane domain; the costimulatory domain selected from the group consisting of: SEQ ID NOs: 36-40, a CD28 costimulatory domain, CD28gg costimulatory domain, a 41-BB costimulatory domain, an OX40 costimulatory domain, and a 2B4 costimulatory domain.

The chimeric antigen receptor can comprise or consist of an amino acid sequence that is 95%, 96%, 97%, 98%, 99% or 100% identical to any of the following sequences, or has 1, 2, 3, 4 or 5 single amino acid substitutions or deletions (preferable deletions are amino terminal or carboxy terminal) compared to, SEQ ID NO: A or B. To calculate percent identity, gapped BLAST can be utilized as described in Altschul et al., Nucleic Acids Res. 25 (17): 3389-3402, 1997. When utilizing BLAST and Gapped BLAST programs, the default parameters of the respective programs (e.g., XBLAST and NBLAST) are be used.

The amino acid sequence of the CAR can be preceded by a signal sequence, for example: MDWIWRILFLVGAATGAHS (SEQ ID NO: 12) or MLLLVTSLLLCELPHPAFLLIP (SEQ ID NO: 62)

Also disclosed are nucleic acid molecules encoding a chimeric antigen receptor, wherein the chimeric antigen receptor comprises: an scFv described herein (e.g., SEQ ID NO: 1); a spacer comprising a sequence selected from the group consisting of: PKSCDKTHTCPPCPDPK (SEQ ID NO: 59) and SEQ ID NOs: 24-34; a transmembrane domain comprising a sequence selected from the group consisting of SEQ ID NOs: 15-23; a costimulatory domain comprising a sequence selected from the group consisting of SEQ ID NOs: 36-40, and a CD3ζ signaling domain comprising SEQ ID NO: 35 (or any of SEQ ID NOs: 50-56).

Also disclosed are immune cells harboring any nucleic acid molecule described herein.

Also disclosed are methods of treating breast cancer (e.g., a breast cancer patient with a HER2-low breast cancer, including: 1) estrogen receptor positive (ER+), HER-2 low breast cancer; and 2) HER2-low, triple-negative breast cancer) the method comprising administering a therapeutically effective amount of immune cells, e.g., NK cells, expressing an EGFR-CAR_sIL15. An EGFR-CAR_sIL15 NK cell or EGFR-CAR_sIL15 T cell can also be used to treat HER2 expressing breast cancer.

### (b) Transmembrane Domain

A CAR disclosed herein can contain a transmembrane domain, which can be a hydrophobic alpha helix that spans the membrane. As used herein, a transmembrane domain refers to any protein structure that is thermodynamically stable in a cell membrane, preferably a eukaryotic cell membrane.

The transmembrane domain of a CAR as provided herein can be a CD28 transmembrane domain having the sequence: FWVLVVVGGVLACYSLLVTVAFIIFWV (SEQ ID NO: 16). Other transmembrane domains can be used including those shown below in Table 1.

TABLE 1
Examples of Transmembrane Domains

| Name | Accession | Length | Sequence |
|---|---|---|---|
| CD3z | JO4132.1 | 21 aa | LCYLLDGILFIYGVILTALFL (SEQ ID NO: 15) |
| CD28 | NM_006139 | 27 aa | FWVLVVVGGVLACYSLLVTVAFIIFWV |
| | | | (SEQ ID NO: 16) |
| CD28(M) | NM_006139 | 28 aa | MFWVLVVVGGVLACYSLLVTVAFIIFWV |
| | | | (SEQ ID NO: 17) |
| CD4 | M35160 | 22 aa | MALIVLGGVAGLLLFIGLGIFF (SEQ ID NO: |
| | | | 18) |
| CD8tm | NM_001768 | 21 aa | IYIWAPLAGTCGVLLLSLVIT (SEQ ID NO: |
| | | | 19) |
| CD8tm2 | NM_001768 | 23 aa | IYIWAPLAGTCGVLLLSLVITLY (SEQ ID NO: |
| | | | 20) |
| CD8tm3 | NM_001768 | 24 aa | IYIWAPLAGTCGVLLLSLVITLYC (SEQ ID |
| | | | NO: 21) |
| 4-1BB | NM_001561 | 27 aa | IISFFLALTSTALLFLLFF LTLRFSVV (SEQ |
| | | | ID NO: 22) |
| NKG2D | NM_007360 | 21 aa | PFFFCCFIAVAMGIRFIIMVA (SEQ ID NO: |
| | | | 23) |

### (c) Spacer Region

A CAR or described herein can include a spacer region located between the EGFR targeting domain (i.e., an EGFR targeted scFv or variant thereof) and the transmembrane domain. Without being bound by theory, the spacer region can function to provide flexibility to the CAR, or domains thereof, or to prevent steric hindrance of the CAR, or domains thereof. A variety of different spacers can be used. Some of them include at least portion of a human Fc region, for example a hinge portion of a human Fc region or a CH3 domain, or variants thereof. For example, a suitable spacer can comprise or consist of the sequence: PKSCDKTHTCPPCPDPK (SEQ ID NO: 59). Table 2 below provides 10 various spacers that can be used in the CARs or polypeptides described herein.

TABLE 2
Examples of Spacers

| Name | Length | Sequence |
|---|---|---|
| a3 | 3 aa | AAA |
| linker | 10 aa | GGGSSGGGSG (SEQ ID NO: 24) |
| IgG4 hinge (S→P) | 12 aa | ESKYGPPCPPCP (SEQ ID NO: 25) |
| (S228P) | | |
| IgG4 hinge | 12 aa | ESKYGPPCPSCP (SEQ ID NO: 26) |
| IgG4 hinge (S228P) + | 22 aa | ESKYGPPCPPCPGGGSSGGGSG (SEQ ID |
| linker | | NO: 27) |
| Also called HL | | |
| CD28 hinge | 39 aa | IEVMYPPPYLDNEKSNGTIIHVKGKHLCPSPL |
| | | FPGPSKP (SEQ ID NO: 28) |
| CD8 hinge-48aa | 48 aa | AKPTTTPAPRPPTPAPTIASQPLSLRPEACRP |
| | | AAGGAVHTRGLDFACD (SEQ ID NO: 29) |
| CD8 hinge-45aa | 45 aa | TTTPAPRPPTPAPTIASQPLSLRPEACRPAAG |
| | | GAVHTRGLDFACD (SEQ ID NO: 30) |
| IgG4 (HL-CH3) | 129 aa | ESKYGPPCPPCPGGGSSGGGSGGQPREPQV |
| Also called: IgG4 | | YTLPPSQEEMTKNQVSLTCLVKGFYPSDIAV |
| (HL-ΔCH2) | | EWESNGQPENNYKTTPPVLDSDGSFFLYSRL |
| (includes S228P in | | TVDKSRWQEGNVFSCSVMHEALHNHYTQKS |
| hinge) | | LSLSLGK (SEQ ID NO: 31) |
| IgG4(L235E, N297Q) | 229 aa | ESKYGPPCPSCPAPEFEGGPSVFLFPPKPKD |
| | | TLMISRTPEVTCVVVDVSQEDPEVQFNWYVD |
| | | GVEVHNAKTKPREEQFQSTYRVVSVLTVLHQ |
| | | DWLNGKEYKCKVSNKGLPSSIEKTISKAKGQP |
| | | REPQVYTLPPSQEEMTKNQVSLTCLVKGFYP |
| | | SDIAVEWESNGQPENNYKTTPPVLDSDGSFF |
| | | LYSRLTVDKSRWQEGNVFSCSVMHEALHNH |
| | | YTQKSLSLSLGK (SEQ ID NO: 32) |
| IgG4(S228P, | 229 aa | ESKYGPPCPPCPAPEFEGGPSVFLFPPKPKD |
| L235E, N297Q) | | TLMISRTPEVTCVVVDVSQEDPEVQFNWYVD |
| | | GVEVHNAKTKPREEQFQSTYRVVSVLTVLHQ |
| | | DWLNGKEYKCKVSNKGLPSSIEKTISKAKGQP |
| | | REPQVYTLPPSQEEMTKNQVSLTCLVKGFYP |
| | | SDIAVEWESNGQPENNYKTTPPVLDSDGSFF |
| | | LYSRLTVDKSRWQEGNVFSCSVMHEALHNH |
| | | YTQKSLSLSLGK (SEQ ID NO: 33) |
| IgG4(CH3) | 107 aa | GQPREPQVYTLPPSQEEMTKNQVSLTCLVKG |
| Also called | | FYPSDIAVEWESNGQPENNYKTTPPVLDSDG |
| IgG4(ΔCH2) | | SFFLYSRLTVDKSRWQEGNVFSCSVMHEALH |
| | | NHYTQKSLSLSLGK (SEQ ID NO: 34) |

Some spacer regions include all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain (called CH3 or ΔCH2) or both a CH3 domain and a CH2 domain. The immunoglobulin derived sequences can include one or more amino acid modifications, for example, 1, 2, 3, 4 or 5 substitutions, e.g., substitutions that reduce off-target binding.

The hinge/linker region can also comprise an IgG4 hinge region having the sequence ESKYGPPCPSCP (SEQ ID NO: 26) or ESKYGPPCPPCP (SEQ ID NO: 25). The hinge/linger region can also comprise the sequence ESKYGPPCPPCP (SEQ ID NO: 25) followed by the linker sequence GGGSSGGGSG (SEQ ID NO: 24) followed by IgG4 CH3 sequence: GQPREPQVYTLPPSQEEMTKNQVSLTCLVKGFYPSDIAVEWESNGQPENNYKTT PPVLDSDGSFFLYSRLTVDKSRWQEGNVFSCSVMHEALHNHYTQKSLSLSLGK (SEQ ID NO: 34). Thus, the entire linker/spacer region can comprise the sequence: ESKYGPPCPPCPGGGSSGGGSGGQPREPQVYTLPPSQEEMTKNQVSLTCLVKGFY PSDIA VEWESNGQPENNYKTTPPVLDSDGSFFLYSRLTVDKSRWQEGNVFSCSV MHEALHNHYTQKSLSLSLGK (SEQ ID NO: 31). In some cases, the spacer has 1, 2, 3, 4, or 5 single amino acid changes (e.g., conservative changes) compared to SEQ ID NO: 31. In some cases, the IgG4 Fc hinge/linker region that is mutated at two positions (L235E; N297Q) in a manner that reduces binding by Fc receptors (FcRs).

### (d) Intracellular Signaling Domains

A CAR constructs described herein contains one or more intracellular signaling domains (e.g., CD3ζ, and optionally one or more co-stimulatory domains), which are the functional end of the receptor. Following antigen recognition, receptors cluster and a signal is transmitted to the cell.

CD3ζ is the cytoplasmic signaling domain of the T cell receptor complex. CD3ζ contains three immunoreceptor tyrosine-based activation motifs (ITAMs), which transmit an activation signal to the cell after the cell is engaged with a cognate antigen. In some cases, CD3ζ provides a primary cell activation signal but not a fully competent activation signal, which requires a co-stimulatory signal.

Accordingly, in some examples, the CAR polypeptides disclosed herein may further comprise one or more co-stimulatory signaling domains in addition to CD3ζ. For example, the co-stimulatory domain CD28 and/or 4-1BB can be used to transmit a proliferative/survival signal together with the primary signaling mediated by CD3ζ. The co-stimulatory domain(s) are located between the transmembrane domain and the CD3ζ signaling domain. Table 3 includes examples of suitable co-stimulatory domains together with the sequence of the CD3ζ signaling domain.

TABLE 3
CD3ζ Domain and Examples of Co-stimulatory Domains

| Name | Accession | Length | Sequence |
|---|---|---|---|
| CD3ζ | J04132.1 | 113 aa | RVKFSRSADAPAYQQGQNQLYNELNLGRREEYD |
| | | | VLDKRRGRDPEMGGKPRRKNPQEGLYNELQKDK |
| | | | MAEAYSEIGMKGERRRGKGHDGLYQGLSTATK |
| | | | DTYDALHMQALPPR (SEQ ID NO: 35) |
| | | | ITAMS 1-3 underlined |
| CD3ζ | | 113 aa | RVKFSRSADAPAYQQGQNQLYNELNLGRREEYD |
| variant | | | VLDKRRGRDPEMGGKPRRKNPQEGLFNELQKDK |
| | | | MAEAFSEIGMKGERRRGKGHDGLFQGLSTATK |
| | | | DTFDALHMQALPPR (SEQ ID NO: 50) |
| CD3ζ | | 113 aa | RVKFSRSADAPAYQQGQNQLYNELNLGRREEYD |
| variant | | | VLDKRRGRDPEMGGKPRRKNPQEGLFNELQKDK |
| | | | MAEAFSEIGMKGERRRGKGHDGLYQGLSTATK |
| | | | DTYDALHMQALPPR (SEQ ID NO: 51) |
| CD3ζ | | 113 aa | RVKFSRSADAPAYQQGQNQLYNELNLGRREEYD |
| variant | | | VLDKRRGRDPEMGGKPRRKNPQEGLYNELQKDK |
| | | | MAEAYSEIGMKGERRRGKGHDGLFQGLSTATK |
| | | | DTFDALHMQALPPR (SEQ ID NO: 52) |
| CD3ζ | | 113 aa | RVKFSRSADAPAYQQGQNQLYNELNLGRREEYD |
| variant | | | VLDKRRGRDPEMGGKPRRKNPQEGLYNELQKDK |
| | | | MAEAFSEIGMKGERRRGKGHDGLFQGLSTATK |
| | | | DTFDALHMQALPPR (SEQ ID NO: 53) |
| CD3ζ | | 113 aa | RVKFSRSADAPAYQQGQNQLYNELNLGRREEYD |
| variant | | | VLDKRRGRDPEMGGKPRRKNPQEGLFNELQKDK |
| | | | MAEAYSEIGMKGERRRGKGHDGLFQGLSTATK |
| | | | DTFDALHMQALPPR (SEQ ID NO: 54) |
| CD3ζ | | 113 aa | RVKFSRSADAPAYQQGQNQLYNELNLGRREEYD |
| variant | | | VLDKRRGRDPEMGGKPRRKNPQEGLFNELQKDK |
| | | | MAEAFSEIGMKGERRRGKGHDGLYQGLSTATK |
| | | | DTFDALHMQALPPR (SEQ ID NO: 55) |
| CD3ζ | | 113 aa | RVKFSRSADAPAYQQGQNQLYNELNLGRREEYD |
| variant | | | VLDKRRGRDPEMGGKPRRKNPQEGLFNELQKDK |
| | | | MAEAFSEIGMKGERRRGKGHDGLFQGLSTATK |
| | | | DTYDALHMQALPPR (SEQ ID NO: 56) |
| CD28 | NM_ | 42 aa | RSKRSRLLHSDYMNMTPRRPGPTRKHYQPYAPP |
| | 006139 | | RDFAAYRS (SEQ ID NO: 36) |
| CD28g | NM_ | 42 aa | RSKRSRGGHSDYMNMTPRRPGPTRKHYQPYAP |
| g* | 006139 | | PRDFAAYRS (SEQ ID NO: 37) |
| 41BB | NM_ | 42 aa | KRGRKKLLYIFKQPFMRPVQTTQEEDGCSCRFP |
| | 001561 | | EEEEGGCEL (SEQ ID NO: 38) |
| OX40 | NM_ | 42 aa | ALYLLRRDQRLPPDAHKPPGGGSFRTPIQEEQA |
| | 003327 | | DAHSTLAKI (SEQ ID NO: 39) |
| 2B4 | NM_ | 120 aa | WRRKRKEKQSETSPKEFLTIYEDVKDLKTRRNHE |
| | 016382 | | QEQTFPGGGSTIYSMIQSQSSAPTSQEPAYTLYS |
| | | | LIQPSRKSGSRKRNHSPSFNSTIYEVIGKSQPKA |
| | | | QNPARLSRKELENFDVYS (SEQ ID NO: 40) |

In some examples, the CD3ζ signaling domain comprises an amino acid sequence that is at least 90%, at least 95%, at least 98% identical to SEQ ID NO: 35. In such instances, the CD3ζ signaling domain has 1, 2, 3, 4, or 5 amino acid changes (preferably conservative substitutions) compared to SEQ ID NO: 35. In other examples, the CD3ζ signaling domain is SEQ ID NO: 35 or a CD3ζ variant (e.g., any of SEQ ID NOs: 50-56).

In various embodiments: the co-stimulatory domain is selected from the group consisting of: a co-stimulatory domain depicted in Table 3 or a variant thereof having 1-5 (e.g., 1 or 2) amino acid modifications, a CD28 or CD28gg co-stimulatory domain or a variant thereof having 1-5 (e.g., 1 or 2) amino acid modifications, a 4-1BB co-stimulatory domain or a variant thereof having 1-5 (e.g., 1 or 2) amino acid modifications and an OX40 co-stimulatory domain or a variant thereof having 1-5 (e.g., 1 or 2) amino acid modifications. In certain embodiments, a 4-1BB co-stimulatory domain or a variant thereof having 1-5 (e.g., 1 or 2) amino acid modifications is present in the CAR polypeptides described herein.

In some embodiments, there are two co-stimulatory domains, for example, a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions). In various embodiments the 1-5 (e.g., 1 or 2) amino acid modification are substitutions. In various embodiments, the co-stimulatory domain is amino terminal to the CD3ζ signaling domain and a short linker consisting of 2-10, e.g., 3 amino acids (e.g., GGG) is can be positioned between the co-stimulatory domain and the CD3ζ signaling domain.

### II. Preparation of CAR NK Cells

In some cases, the CAR can be produced using a vector in which the CAR open reading frame is followed by a T2A ribosome skip sequence and a truncated EGFR (EGFRt), which lacks the cytoplasmic signaling tail, or a truncated CD19R (also called CD19t). In this arrangement, co-expression of EGFRt or CD19t provides an inert, non-immunogenic surface marker that allows for accurate measurement of gene modified cells, and enables positive selection of gene-modified cells, as well as efficient cell tracking of the therapeutic immune cells in vivo following adoptive transfer. Efficiently controlling proliferation to avoid cytokine storm and off-target toxicity is an important hurdle for the success of immune cell immunotherapy. The EGFRt or the CD19t incorporated in the CAR lentiviral vector can act as suicide gene to ablate the CAR+ NK cells in cases of treatment-related toxicity.

The CAR, including the ODD, can be followed by a ribosomal skip sequence (e.g., GSGATNFSLLKQAGDVEENPGP (SEQ ID NO: 14) or LEGGGEGRGSLLTCGDVEENPGPR; SEQ ID NO: 45) and a truncated CD19 receptor (also called CD19t) having a sequence that is at least 90%, at least 95%, at least 98% identical to or identical to:

(SEQ ID NO: 64)
MPPPRLLFFLLFLTPMEVRPEEPLVVKVEEGDNAVLQCLKGTSDG
PTQQLTWSRESPLKPFLKLSLGLPGLGIHMRPLAIWLFIFNVSQQ
MGGFYLCQPGPPSEKAWQPGWTVNVEGSGELFRWNVSDLGGLGCG
LKNRSSEGPSSPSGKLMSPKLYVWAKDRPEIWEGEPPCVPPRDSL
NQSLSQDLTMAPGSTLWLSCGVPPDSVSRGPLSWTHVHPKGPKSL
LSLELKDDRPARDMWVMETGLLLPRATAQDAGKYYCHRGNLTMSF
HLEITARPVLWHWLLRTGGWKVSAVTLAYLIFCLCSLVGILHLQR
ALVLRRKR.

Any CAR described herein can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region can be inserted into an expression vector and used to transform a suitable expression host cell line. A suitable host cell line includes, for example, an NK cell. An expression vector encoding a CAR or polypeptide described herein can be a viral vector. Suitable viral vectors, including lentiviral vectors, are known in the art and can be used in any of the methods described herein. In some aspects, any of the transduced immune cells described herein can be autologous or allogenic. For example, suitable cell populations can include allogenic NK cells, autologous NK cells, allogenic NK cells and autogenic NK cells.

The CAR can be transiently expressed in an NK cell population by an mRNA encoding the CAR. The mRNA can be introduced into immune cells by electroporation (Wiesinger et al. 2019 Cancers (Basel) 11:1198).

Disclosed herein, amongst other things, are methods of making any CAR NK ll described herein by introducing into a NK cell a vector comprising a nucleotide sequence encoding the CAR. Disclosed herein, amongst other things, are methods of making a population of NK cells comprising a nucleic acid encoding any CAR or polypeptide described herein.

### IV. Treatment of Cancer

Aspects of the present disclosure provide methods for treating a subject having breast cancer by administering immune cells expressing a CAR described here.

### (a) Subjects

A subject to be treated by the methods described can be a human patient having a breast cancer with low or no HER2 expression, e.g., breast cancer that is HER2 immunohistochemistry (IHC) 1+ or 2+ with negative fluorescence in situ hybridization (FISH).

### (b) Treatment Regimens

Aspects of the present disclosure provide methods of treating breast cancer comprising administering a lymphodepletion treatment (e.g., cyclophosphamide) in combination with CAR immune cells, which can be administered locally or systemically. Generally, the methods include administering a therapeutically effective amount of a population of CAR NK cells as described herein, to a subject who is in need of, or who has been determined to be in need of, such treatment. The population of EGFR CAR_sIL-15 immune cells in all compositions and methods disclosed herein can be autologous or allogenic.

Any subject suitable for the treatment methods described herein can receive a lymphodepleting therapy to reduce or deplete the endogenous lymphocytes of the subject. Lymphodepletion refers to the destruction of endogenous lymphocytes, which is commonly used prior to immunotransplantation and immunotherapy. Lymphodepletion can be achieved by administering a lymphodepleting agent and/or irradiation (e.g., stereotactic radiation). A lymphodepleting agent can be any molecule capable of reducing, depleting, or eliminating endogenous lymphocytes when administered to a subject. In some examples, the lymphodepleting agents are cytotoxic agents that specifically kill lymphocytes. Non-limiting examples of lymphodepleting agents include cyclophosphamide, fludarabine, gemcitabine, methotrexate, doxorubicin, and etopside phosphate. In some cases the lymphodepletion treatment is non-myeloablative.

Methods described herein can include a conditioning regimen comprising a single lymphodepleting agent (e.g., cyclophosphamide) or multiple lymphodepleting agents (e.g., cyclophosphamide and fludarabine). The subject to be treated by the methods described herein can receive one or more doses of the one or more lymphodepleting agents for a period suitable for reducing or depleting the endogenous lymphocytes of the subject (e.g., 1-5 days).

The subject can then be administered any of the CAR immune cells described herein after administration of the lymphodepleting therapy as described herein. For example, the one or more lymphodepleting agents can be administered to the subject 1-5 days (e.g., 1, 2, 3, 4, or 5 days) prior to administering the CAR immune cells.

Methods described herein can include redosing the subject with CAR immune cells. In some examples, the subject is administered a lymphodepleting treatment prior to redosing of the anti-CAR immune cells. Each dose of the CAR immune cells can be the same or the doses can be ascending or descending.

Methods described herein can include redosing the subject with CAR immune cells. In some examples, the subject is administered 3-6 doses of the CAR immune cells, each of which is administered 1-15 days after the prior dose. Each dose of CAR immune cells can be the same or the doses can be ascending or descending.

### (c) Administration

An effective amount of a therapy (e.g., EGFR-CAR_sIL15 NK cells) can be administered to a subject (e.g., a human) in need of the treatment via any suitable route (e.g., administered locally or systemically to a subject). Suitable modes of administration include injection, infusion, instillation, or ingestion. Injection includes, without limitation, intravenous, intramuscular, intra-arterial, intrathecal, intraventricular, intradermal, intraperitoneal, and subcutaneous injection and infusion.

An effective amount, or therapeutically effective amount, refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of treatment, the nature of concurrent therapy, if any, the specific route of administration and like factors within the knowledge and expertise of the health practitioner. The amelioration of one symptom associated with the condition, cancer, or disease is enough to confer therapeutic effect on the subject. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Disclosed herein, amongst other things, methods of administering to a subject in need thereof (e.g., a subject having breast cancer), a therapeutic amount of any disclosed cell population comprising a nucleic acid encoding an EGFR-CAR_sIL15 described herein. Disclosed herein, amongst other things, methods of administering to a subject in need thereof (e.g., a subject having breast cancer), a therapeutic amount of any disclosed cell population expressing any an EGFR-CAR_sIL15 described herein.

The materials, methods, and examples are illustrative only and not intended to be limiting. All publications, patent applications, patents, sequences, database entries, and other references mentioned herein are incorporated by reference in their entirety for any and all purposes.

Other features and advantages of the described compositions and methods will be apparent from the following detailed description and figures, and from the claims.

### DESCRIPTION OF DRAWINGS

FIG. 1 depicts expression of EGFR and HER2 in breast cancer. EGFR and HER2 immunohistochemistry of tumor tissue from patients with primary breast cancer using an anti-human EGFR antibody. Representative data of 3 out of 24 patients are presented.

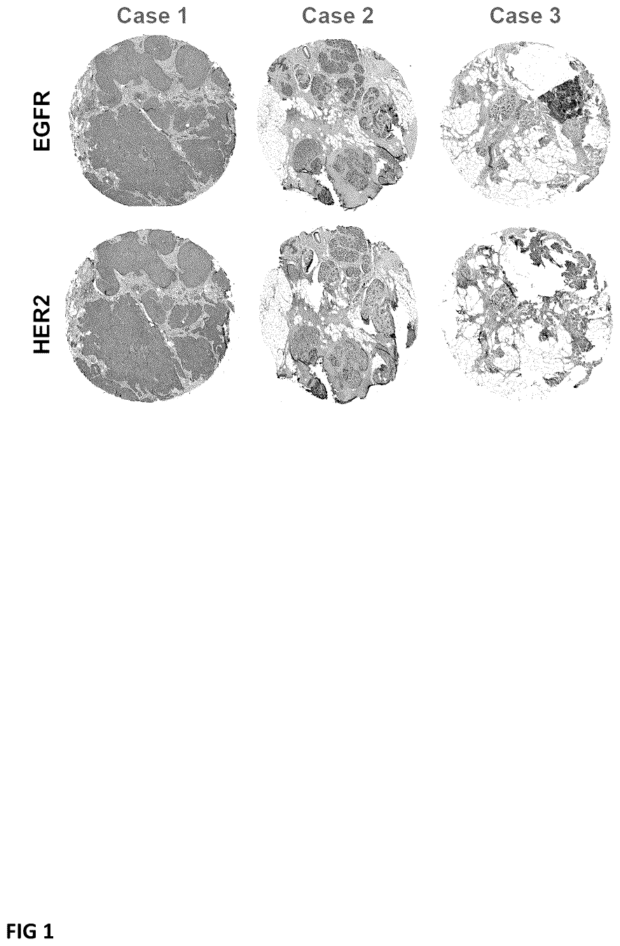

FIG. 2 depicts a schematic of the structure of an EGFR-CAR_sIL15 CAR. SP, signal peptide; TM, transmembrane domain; ODD, oxygen dependent degradation domain of HIF1a; T2A, ribosomal skip sequence; Codon Opm I15, Codon-optimized soluble IL-15; P2A, porcine teschovirus 2A ribosomal skip sequence; tCD19, truncated CD19 receptor.

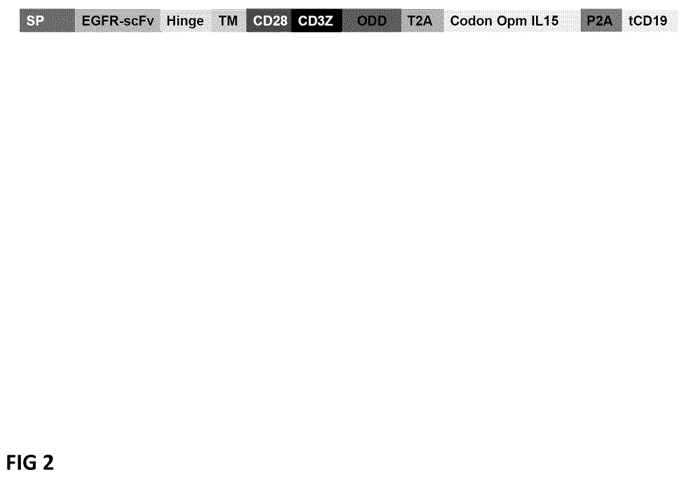

FIG. 3 depicts the results of an analysis of expression of EGFR CAR_sIL15 and control vector transduction of UCB-derived NK cells as assessed by tCD9 expression.

FIGS. 4A-4B depict the result of an in vitro functional evaluation of EGFR-CAR_sIL15 NK cells. (A-B) Cytotoxicity against MDA-MB-231 and MCF-7 (EGFR+) cells (A) and A2780 (EGFR−) (B) cells were tested after co-culture of EGFR_sIL15-CAR NK cells, NK cells expressing IL-15 (sIL15NK), or unmodified NK cells at different E:T ratios analyzed using ⁵¹Cr assay.

FIGS. 5A-5B depict the results of an in vitro functional evaluation of EGFR-CAR sIL15 NK cells under hypoxia. (A) Cytotoxicity against MDA-MB-231 (EGFR+) cells was tested after co-culture of EGFR_sIL15-CAR NK cells or unmodified NK cells under hypoxia or normoxia condition at different effector (E):target (T) ratios analyzed using ⁵¹Cr assay. (B) The cytotoxicity was analyzed by microscope under hypoxia conditions at E:T=2:1. PI was used to stain dead cells (bright red).

FIGS. 6A-6D depict the results of a study showing that EGFR-CAR_sIL15 NK cells improve tumor rejection and prolong survival of mice bearing breast tumor that metastasized to lung. (A) Schematic of metastatic breast cancer model (5×10⁵ cells). (B) Time-lapse luciferase imaging of breast cancer tumor mice with indicated treatments (4×10⁶ CAR NK cells/infusion). (C) Quantification of bioluminescence images. (D) Survival analysis of the breast tumor model that metastasized to the lung.

### DETAILED DESCRIPTION

### Examples

The invention is further described in the following examples, which do not limit the scope of the invention described in the claims.

### Example 1: Expansion and Transduction of Natural Killer Cells

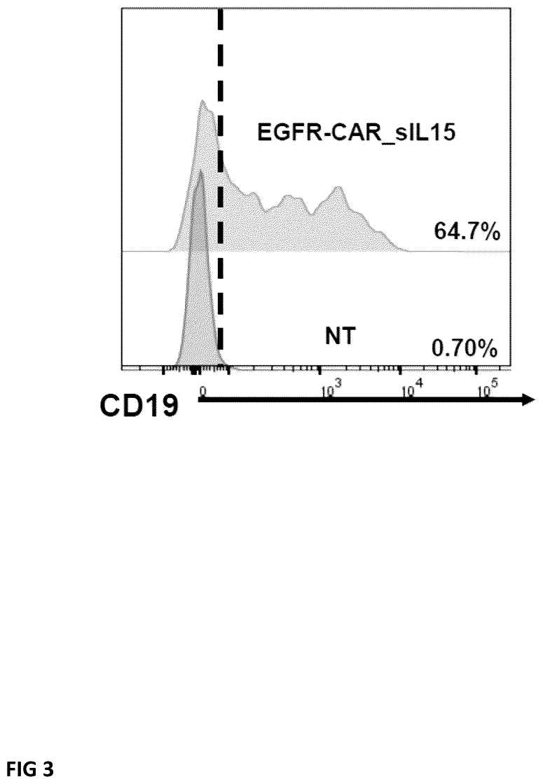

We used an efficient retrovirus-based transduction system to optimize the manufacturing of CAR NK cells. Briefly, we expanded UCB NK cells on a K562 feeder cell layer expressing membrane-bound interleukin-21 (IL-21) and 4-1BB ligand (4-1BBL). The expanded NK cells were infected with a replication-defective retroviral vector expressing EGFR-CAR_sIL15 prior to further expansion. This platform improved transduction efficiency and survival of NK cells¹¹. Using our system, we achieved ˜60-70% transduction of human NK cells, which is comparable to the transduction of T cells with the same retrovirus expressing a CAR (data not shown). As we incorporated sIL-15 into our vector to enhance survival of CAR NK cells in vivo, for the control, we also generated a vector expressing sIL-15 and tEGFR without the EGFR-CAR (FIG. 3).

### Example 2: Assessment of Cytotoxicity

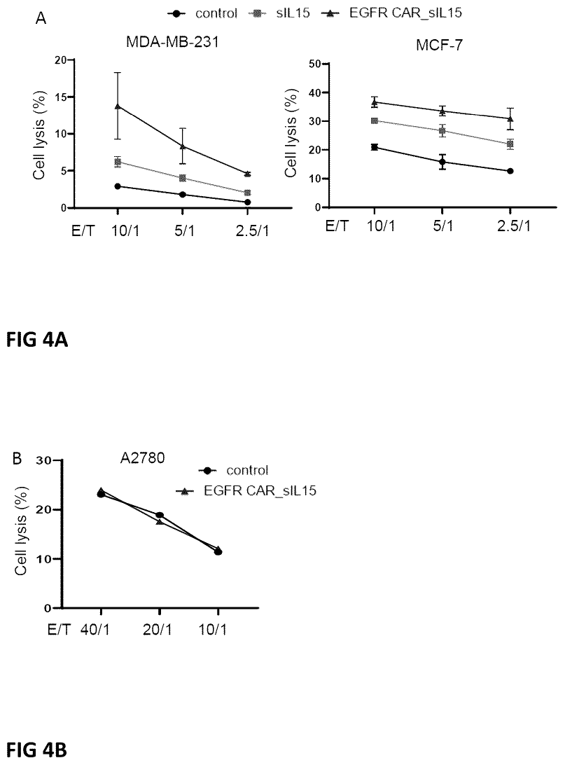

We tested EGFR-CAR_sIL15 NK cells for cytotoxicity against the two MDA-MB-231 and MCF-7 BC cell lines, both of which are EGFR⁺ and HER2⁻. To do this, we co-cultured EGFR-CAR_sIL15 NK cells, NK cells only expressing sIL15 (sIL15 NK) and unmodified control NK cells, with tumor cells at different effector:target (E:T) ratios. We analyzed cytotoxicity using ⁵¹Cr assay. EGFR-CAR_sIL15 NK cells killed BC cells substantially better than sIL15 NK and unmodified control NK cells (FIG. 4A). We also measured the cytotoxicity against A2780 cells, which are EGFR⁻. There was no cytotoxicity difference between unmodified control NK cells and EGFR-CAR_sIL15 NK cells targeting EGFR-cells (FIG. 4B).

### Example 3: Assessment of Activity in Hypoxia and Normoxia

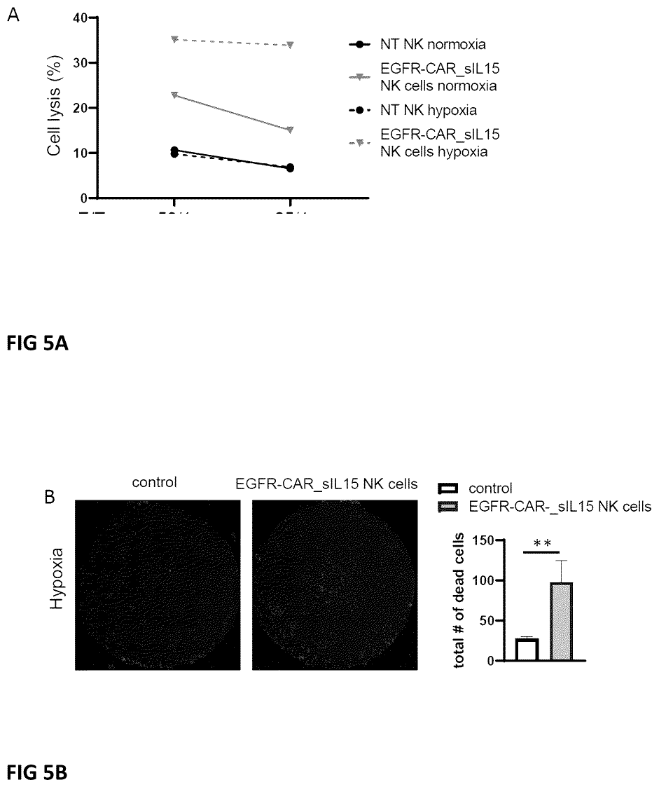

Hypoxia is a characteristic feature shared by most advanced solid tumors, which promotes a heterogeneous landscape of oxygen levels and metabolic activities. It has been reported that hypoxia is a physical cue for licensing CAR-T cell or immune cell activation, specifically in the tumor microenvironment. Therefore, we tested EGFR-CAR_sIL15 NK cells for cytotoxicity against the EGFR MDA-MB-231 BC cell line under hypoxia conditions. Due to the ODD in the construct, EGFR-CAR_sIL15 NK cells killed BC cells substantially better under the hypoxia condition than that under the normoxia condition. In contrast, there is no difference for nontransduced NK cells with a very low level of cytotoxicity at both the normoxia and hypoxia conditions. The data indicated that NK cells engineered with the EGFR-CAR_sIL15 vector fused with ODD could be safe and effective in targeting solid tumors (FIGS. 5A-5B).

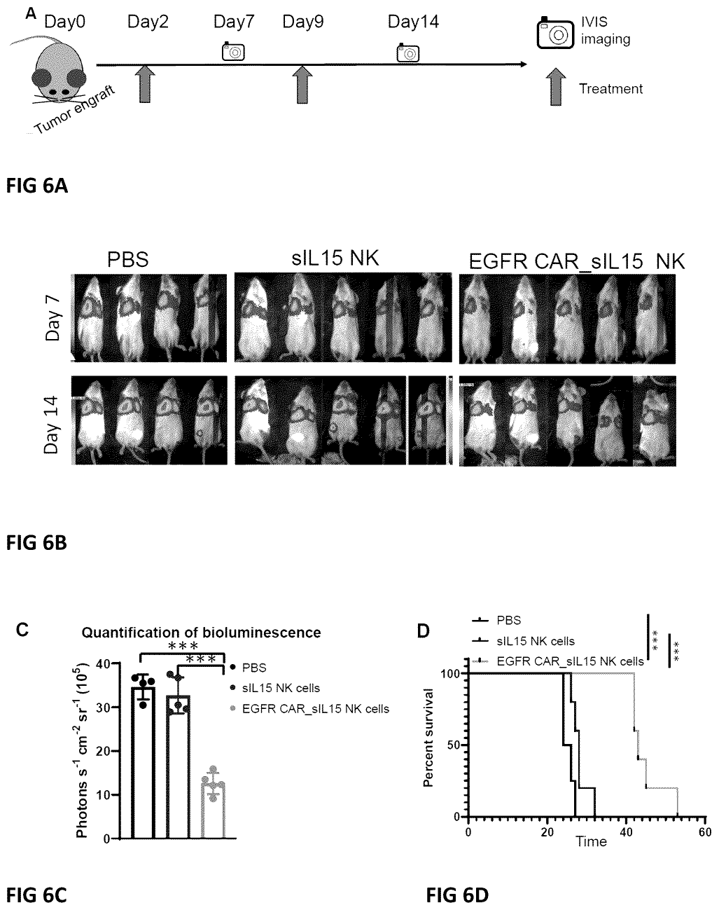

To test the efficacy of EGFR-CAR_sIL15 NK cells in vivo, we first injected (iv) 5×10⁵ MDA-MB-231 cells expressing luc per mouse to establish a metastatic BC mouse model (FIG. 6A). We manufactured frozen, off-the-shelf EGFR-CAR_sIL15 NK cells and sIL15 control NK cells from a same UCB unit. Two days after tumor implantation, we randomized mice into three groups that received three cycles of PBS, off-the-shelf frozen EGFR-CAR_sIL15 NK cells, or control sIL15 NK cells given intravensously. We monitored tumor progression using luc-based imaging starting 7 days post-tumor implantation. EGFR-CAR_sIL15 NK cells were significantly more effective than the other two groups at improving the rejection of breast tumors and survival (FIGS. 6B-6D).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A nucleic acid molecule encoding a fusion protein comprising a chimeric antigen receptor and an oxygen-dependent degradation domain (ODD), wherein the chimeric antigen receptor comprises:
(i) an scFv that binds human EGFR;
(ii) a spacer domain;
(iii) a transmembrane domain;
(iv) a costimulatory domain; and
(v) a CD3ζ signaling domain;
wherein the scFv comprises: a light chain CDR1 comprising RSSQNIVHNNGITYLE (SEQ ID NO: 4), a light chain CDR2 comprising KVSDRFS (SEQ ID NO: 5), a light chain CDR3 comprising FQGSHIPPT (SEQ ID NO: 6), a heavy chain CDR1 comprising SYWMH (SEQ ID NO: 7), a heavy chain CDR2 comprising NIYPGSGGTNYAEKFKN (SEQ ID NO: 8), and a heavy chain CDR3 comprising SGGPYFFDY (SEQ ID NO: 9).

2. (canceled)

3. The nucleic acid molecule of claim 1, wherein the ODD is a human HIF1α ODD.

4. The nucleic acid molecule of claim 3, wherein the ODD comprises or consists of
(SEQ ID NO: 10)
APAAGDTIISLDFGSNDTETDDQQLEEVPLYNDVMLPSPNEKLQN
INLAMSPLPTAETPKPLRSSADPALNQEVALKLEPNPESLELSFT
MPQIQDQTPSPSDGSTRQSSPEPNSPSEYCFYVDSDMVNEFKLEL
VEKLFAEDTEAKNPFSTQDTDLDLEMLAPYIPMDDDFQLRSFDQL
SPLESSSASPESASPQSTVTVFQ.

5. The nucleic acid molecule of claim 1, wherein the scFv comprises a VL domain comprising: DILMTQSPLSLPVSLGDQASISCRSSQNIVHNNGITYLEWYLQRPGQSPKLLIYKVSDRFS GVPDRFSGSGSGTDFTLKISRVEAEDLGIYYCFQGSHIPPTFGGGTKLEIKRAA (SEQ ID NO: 2) with no more than 10, 5, 4, 3, 2 or 1 single amino acid substitutions followed by a VH domain comprising or consisting of QVQLQQSGSEMARPGASVKLPCKASGDTFTSYWMHWVKQRHGHGPEWIGNIYPGSGG TNYAEKFKNKVTLTVDRSSRTVYMHLSRLTSEDSAVYYCTRSGGPYFFDYWGQGTTLT VSS (SEQ ID NO: 3) with no more than 10, 5, 4, 3, 2 or 1 single amino acid substitutions; optionally wherein the amino acid substitutions are not in the CDRs.

6. The nucleic acid molecule of claim 1, where the scFv comprises a VL domain comprising or consisting of an amino acid sequence at least 95%, 98% or 99% identical to: DILMTQSPLSLPVSLGDQASISCRSSQNIVHNNGITYLEWYLQRPGQSPKLLIYKVSDRFS GVPDRFSGSGSGTDFTLKISRVEAEDLGIYYCFQGSHIPPTFGGGTKLEIKRAA (SEQ ID NO: 2) with no more than 10, 5, 4, 3, 2 or 1 single amino acid substitutions followed by a VH domain comprising or consisting of an amino acid sequence at least 95%, 98% or 99% identical to QVQLQQSGSEMARPGASVKLPCKASGDTFTSYWMHWVKQRHGHGPEWIGNIYPGSGG TNYAEKFKNKVTLTVDRSSRTVYMHLSRLTSEDSAVYYCTRSGGPYFFDYWGQGTTLT VSS (SEQ ID NO: 3) with no more than 10, 5, 4, 3, 2 or 1 single amino acid substitutions; optionally wherein the amino acid substitutions are not in the CDRs.

7. (canceled)

8. The nucleic acid molecule of claim 1, wherein the chimeric antigen receptor comprises: the spacer comprises or consists of a sequence selected from the group consisting of: SEQ ID NOs: 59 and 24-34; the transmembrane domain comprises or consists of a sequence selected from the group consisting of SEQ ID NOs: 15-23; the costimulatory domain comprises or consists of a sequence from the group consisting of SEQ ID NOs: 36-40, and the CD3ζ signaling domain comprises or consists of SEQ ID NO: 35.

9. The nucleic acid molecule of claim 1, wherein the nucleic acid molecule further encodes human IL-15 or a soluble variant of human IL-15.

10. The nucleic acid molecule of claim 1, wherein the nucleic acid molecule further encodes human IL-15 or a soluble variant of human IL-15 that is co-expressed with the fusion protein.

11. The nucleic acid molecule of claim 9, wherein the soluble variant of IL-15 comprises or consists of: GIHVFILGCFSAGLPKTEANWVNVISDLKKIEDLIQSMHIDATLYTESDVHPSCKVTAMK CFLLELQVISLESGDASIHDTVENLIILANNSLSSNGNVTESGCKECEELEEKNIKEFLQSF VHIVQMFINTS (SEQ ID NO:11).

12.-13. (canceled)

14. The nucleic acid molecule of claim 1 encoding an amino acid sequence comprising or consisting of the amino acid sequence:
(SEQ ID NO: 57 [[A]])
DILMTQSPLSLPVSLGDQASISCRSSQNIVHNNGITYLEWYLQRP
GQSPKLLIYKVSDRFSGVPDRFSGSGSGTDFTLKISRVEAEDLGI
YYCFQGSHIPPTFGGGTKLEIKRAAGGGGSGGGGSGGGGSQVQLQ
QSGSEMARPGASVKLPCKASGDTFTSYWMHWVKQRHGHGPEWIGN
IYPGSGGTNYAEKFKNKVTLTVDRSSRTVYMHLSRLTSEDSAVYY
CTRSGGPYFFDYWGQGTTLTVSSPKSCDKTHTCPPCPDPKFWVLV
VVGGVLACYSLLVTVAFIIFWVRSKRSRLLHSDYMNMTPRRPGPT
RKHYQPYAPPRDFAAYRSRVKFSRSADAPAYQQGQNQLYNELNLG
RREEYDVLDKRRGRDPEMGGKPRRKNPQEGLYNELQKDKMAEAYS
EIGMKGERRRGKGHDGLYQGLSTATKDTYDALHMQALPPRAPAAG
DTIISLDFGSNDTETDDQQLEEVPLYNDVMLPSPNEKLQNINLAM
SPLPTAETPKPLRSSADPALNQEVALKLEPNPESLELSFTMPQIQ
DQTPSPSDGSTRQSSPEPNSPSEYCFYVDSDMVNEFKLELVEKLF
AEDTEAKNPFSTQDTDLDLEMLAPYIPMDDDFQLRSFDQLSPLES
SSASPESASPQSTVTVFQ
with no more than 10, 5, 4, 3, 2, 1 or no single amino acid substitutions or the amino acid sequence:
(SEQ ID NO: 58[B])
DILMTQSPLSLPVSLGDQASISCRSSQNIVHNNGITYLEWYLQRP
GQSPKLLIYKVSDRFSGVPDRFSGSGSGTDFTLKISRVEAEDLGI
YYCFQGSHIPPTFGGGTKLEIKRAAGGGGSGGGGSGGGGSQVQLQ
QSGSEMARPGASVKLPCKASGDTFTSYWMHWVKQRHGHGPEWIGN
IYPGSGGTNYAEKFKNKVTLTVDRSSRTVYMHLSRLTSEDSAVYY
CTRSGGPYFFDYWGQGTTLTVSSPKSCDKTHTCPPCPDPKFWVLV
VVGGVLACYSLLVTVAFIIFWVRSKRSRLLHSDYMNMTPRRPGPT
RKHYQPYAPPRDFAAYRSRVKFSRSADAPAYQQGQNQLYNELNLG
RREEYDVLDKRRGRDPEMGGKPRRKNPQEGLYNELQKDKMAEAYS
EIGMKGERRRGKGHDGLYQGLSTATKDTYDALHMQALPPRAPAAG
DTIISLDFGSNDTETDDQQLEEVPLYNDVMLPSPNEKLQNINLAM
SPLPTAETPKPLRSSADPALNQEVALKLEPNPESLELSFTMPQIQ
DQTPSPSDGSTRQSSPEPNSPSEYCFYVDSDMVNEFKLELVEKLF
AEDTEAKNPFSTQDTDLDLEMLAPYIPMDDDFQLRSFDQLSPLES
SSASPESASPQSTVTVFQAEGRGSLLTCGDVEENPGPMYRMQLLS
CIALSLALVTNSGIHVFILGCFSAGLPKTEANWVNVISDLKKIED
LIQSMHIDATLYTESDVHPSCKVTAMKCFLLELQVISLESGDASI
HDTVENLIILANNSLSSNGNVTESGCKECEELEEKNIKEFLQSFV
HIVQMFINTS
with no more than 10, 5, 4, 3, 2, 1 or no single amino acid substitutions.

15. An immune cell harboring a nucleic acid of claim 1.

16. The immune cell of claim 15, wherein the immune cell is selected from the group consisting of: a T cell, an NK cell, an NKT cell, a macrophage, and a gamma delta T cell.

17. The immune cell of claim 15, wherein the immune cell expresses human IL-15 or soluble variant thereof.

18. The immune cell of claim 17, wherein the soluble variant of IL-15 comprises or consists of:
(SEQ ID NO: 11)
GIHVFILGCFSAGLPKTEANWVNVISDLKKIEDLIQSMHIDATLY
TESDVHPSCKVTAMKCFLLELQVISLESGDASIHDTVENLIILAN
NSLSSNGNVTESGCKECEELEEKNIKEFLQSFVHIVQMFINTS.

19. The immune cell of claim 18, wherein the immune cell is an NK cell.

20. A method for treating breast cancer comprising administering a composition comprising an immune cell of claim 1.

21. A fusion protein comprising a chimeric antigen receptor and an oxygen-dependent degradation domain (ODD), wherein the chimeric antigen receptor comprises:
(i) an scFv that binds human EGFR;
(ii) a spacer domain;
(iii) a transmembrane domain;
(iv) a costimulatory domain; and
(v) a CD3 ζ signaling domain;
wherein the scFv comprises: a light chain CDR1 comprising RSSQNIVHNNGITYLE (SEQ ID NO: 4), a light chain CDR2 comprising KVSDRFS (SEQ ID NO: 5), a light chain CDR3 comprising FQGSHIPPT (SEQ ID NO: 6), a heavy chain CDR1 comprising SYWMH (SEQ ID NO: 7), a heavy chain CDR2 comprising NIYPGSGGTNYAEKFKN (SEQ ID NO: 8), and a heavy chain CDR3 comprising SGGPYFFDY (SEQ ID NO: 9).

22.-23. (canceled)

24. The fusion protein of claim 21, wherein the ODD comprises or consists of APAAGDTIISLDFGSNDTETDDQQLEEVPLYNDVMLPSPNEKLQNINLAMSPLPTAETPK PLRSSADPALNQEVALKLEPNPESLELSFTMPQIQDQTPSPSDGSTRQSSPEPNSPSEYCFY VDSDMVNEFKLELVEKLFAEDTEAKNPFSTQDTDLDLEMLAPYIPMDDDFQLRSFDQLS PLESSSASPESASPQSTVTVFQ (SEQ ID NO: 10).

25. The fusion protein of claim 21, wherein the scFv comprises a VL domain comprising: DILMTQSPLSLPVSLGDQASISCRSSQNIVHNNGITYLEWYLQRPGQSPKLLIYKVSDRFS GVPDRFSGSGSGTDFTLKISRVEAEDLGIYYCFQGSHIPPTFGGGTKLEIKRAA (SEQ ID NO: 2) with no more than 10, 5, 4, 3, 2 or 1 single amino acid substitutions followed by a VH domain comprising or consisting of QVQLQQSGSEMARPGASVKLPCKASGDTFTSYWMHWVKQRHGHGPEWIGNIYPGSGG TNYAEKFKNKVTLTVDRSSRTVYMHLSRLTSEDSAVYYCTRSGGPYFFDYWGQGTTLT VSS (SEQ ID NO: 3) with no more than 10, 5, 4, 3, 2 or 1 single amino acid substitutions; optionally wherein the amino acid substitutions are not in the CDRs.

26. The fusion protein of claim 21, wherein the fusion protein comprises or consists of the amino acid sequence:
(SEQ ID NO: 57 [A]])
DILMTQSPLSLPVSLGDQASISCRSSQNIVHNNGITYLEWYLQRP
GQSPKLLIYKVSDRFSGVPDRFSGSGSGTDFTLKISRVEAEDLGI
YYCFQGSHIPPTFGGGTKLEIKRAAGGGGSGGGGSGGGGSQVQLQ
QSGSEMARPGASVKLPCKASGDTFTSYWMHWVKQRHGHGPEWIGN
IYPGSGGTNYAEKFKNKVTLTVDRSSRTVYMHLSRLTSEDSAVYY
CTRSGGPYFFDYWGQGTTLTVSSPKSCDKTHTCPPCPDPKFWVLV
VVGGVLACYSLLVTVAFIIFWVRSKRSRLLHSDYMNMTPRRPGPT
RKHYQPYAPPRDFAAYRSRVKFSRSADAPAYQQGQNQLYNELNLG
RREEYDVLDKRRGRDPEMGGKPRRKNPQEGLYNELQKDKMAEAYS
EIGMKGERRRGKGHDGLYQGLSTATKDTYDALHMQALPPRAPAAG
DTIISLDFGSNDTETDDQQLEEVPLYNDVMLPSPNEKLQNINLAM
SPLPTAETPKPLRSSADPALNQEVALKLEPNPESLELSFTMPQIQ
DQTPSPSDGSTRQSSPEPNSPSEYCFYVDSDMVNEFKLELVEKLF
AEDTEAKNPFSTQDTDLDLEMLAPYIPMDDDFQLRSFDQLSPLES
SSASPESASPQSTVTVFQ
with no more than 10, 5, 4, 3, 2, 1 or no single amino acid substitutions.
